(19) United States
(12) Patent Application Publication
(10) Pub. No.: US 2017/0051267 A1
(43) Pub. Date: Feb. 23, 2017

(54) NOVEL ALPHA-GALACTOSIDASE A DERIVATIVES

(71) Applicant: Research Foundation of the City University of New York, New York, NY (US)

(72) Inventor: David Calhoun, Leonia, NJ (US)

(21) Appl. No.: 15/243,637

(22) Filed: Aug. 22, 2016

Related U.S. Application Data
(60) Provisional application No. 62/207,856, filed on Aug. 20, 2015.
(60) Provisional application No. 62/207,849, filed on Aug. 20, 2015.

(51) Int. Cl. C12N 9/40

(52) U.S. Cl. CPC C12N 9/2465; C12Y 302/01022; A61K 38/00

(57) ABSTRACT

Fabry disease is a rare (incidence approximately 1 in 20,000) X-linked inborn error of glycolipid metabolism caused by a deficiency of the lysosomal enzyme, α-galactosidase A, that leads to early death in affected males due to occlusive disease of the heart, kidney, and brain. The present invention provides a modified alpha-Galactosidase A enzyme derivative with improved stability and catalytic properties. As a result of the present invention, an effective therapeutic effect is achieved with a lower dose of enzyme infused to the patients.

### CROSS-REFERENCE TO RELATED APPLICATIONS

This application claims priority to U.S. Provisional Application No. 62/207,856 filed Aug. 20, 2015, and Provisional Application No. 62/207,849 filed Aug. 20, 2015, the contents of which are incorporated herein by reference in their entirety.

### BACKGROUND OF THE INVENTION

Fabry disease is a rare (incidence approximately 1 in 20,000) X-linked inborn error of glycolipid metabolism caused by a deficiency of the lysosomal enzyme, α-galactosidase A, that leads to early death in affected males due to occlusive disease of the heart, kidney, and brain. Clinical trials of enzyme replacement therapy for Fabry disease patients revealed clinical efficacy. However, a significant fraction of the patients developed antibodies even though these preliminary clinical trials involved a single infusion of enzyme.

Accordingly, there exists a long standing need to provide a more efficient delivery system to make it possible to achieve an effective therapeutic effect with a lower dose of enzyme infused to the patients.

### BRIEF SUMMARY OF THE INVENTION

The present invention provides a modified alpha-Galactosidase A enzyme derivative with improved stability and catalytic properties.

In one embodiment, the invention provides a modified alpha-Galactosidase A polypeptide of SEQ ID NO. 1 having at least one mutation selected from E203C/N, Y207R/W, Y134W, and R227W.

In another embodiment, the invention provides a method of treating Fabry disease by administering a modified alpha-Galactosidase A polypeptide of SEQ ID NO. 1 having at least one mutation selected from E203C/N, Y207R/W, Y134W, and R227W; to a subject in need thereof.

In one embodiment, the present invention provides a modified alpha-galactosidase A polypeptide of SEQ ID NO. 1 having at least one mutation selected from E48C, L275C, R301C, H302C, R49C, S304C, G361C, P362C, N408C, P409C, D234C, S235C, N272C, F273C, G274C, S276C, W277C, E358C, I359C, and G360C.

In another embodiment, the invention provides a method of treating Fabry disease by administering a modified alpha-galactosidase A polypeptide of SEQ ID NO. 1 having at least one mutation selected from E48C, L275C, R301C, H302C, R49C, S304C, G361C, P362C, N408C, P409C, D234C, S235C, N272C, F273C, G274C, S276C, W277C, E358C, I359C, and G360C; to a subject in need thereof.

In another embodiment, the invention provides a method of treating Fabry disease by administering a modified dimeric alpha-galactosidase A polypeptide of polypeptide monomers of SEQ ID NO. 1 having at least one mutation selected from E48C, L275C, R301C, H302C, R49C, S304C, G361C, P362C, N408C, P409C, D234C, S235C, N272C, F273C, G274C, S276C, W277C, E358C, I359C, and G360C to a subject in need thereof.

In another embodiment, the invention provides a stabilized dimeric alpha-galactosidase A polypeptide having polypeptide monomers of SEQ ID NO. 1 having at least one mutation selected from E48C, L275C, R301C, H302C, R49C, S304C, G361C, P362C, N408C, P409C, D234C, S235C, N272C, F273C, G274C, S276C, W277C, E358C, I359C, and G360C.

In another embodiment, the invention provides a stabilized alpha-galactosidase A dimer including a dimeric polypeptide having monomers an alpha-galactosidase A polypeptide of SEQ ID NO. 1 having at least one mutation selected from E203C/N, Y207R/W, Y134W, R227W, E48C, L275C, R301C, H302C, R49C, S304C, G361C, P362C, N408C, P409C, D234C, S235C, N272C, F273C, G274C, S276C, W277C, E358C, I359C, and G360C; wherein each monomer of the dimer is covalently linked by at least one disulfide bond.

In another embodiment, the invention provides a stabilized alpha-galactosidase A dimer including a dimeric protein having monomers including an alpha-galactosidase A polypeptide of SEQ ID NO. 1 having at least one mutation selected E203C/N, Y207R/W, Y134W, R227W, E48C, L275C, R301C, H302C, R49C, S304C, G361C, P362C, N408C, P409C, D234C, S235C, N272C, F273C, G274C, S276C, W277C, E358C, I359C, and G360C; wherein each monomer of the dimer is covalently linked by at least one disulfide bond.

In another embodiment, the invention provides a method of treating Fabry disease by administering a stabilized alpha-galactosidase A dimer including a dimeric protein having monomers of an alpha-galactosidase A polypeptide of SEQ ID NO. 1 having at least one mutation selected from E203C/N, Y207R/W, Y134W, R227W, E48C, L275C, R301C, H302C, R49C, S304C, G361C, P362C, N408C, P409C, D234C, S235C, N272C, F273C, G274C, S276C, W277C, E358C, I359C, and G360C; wherein each monomer of the dimer is covalently linked by at least one disulfide bond.

As a result of the present invention, an effective therapeutic effect is achieved with a lower dose of enzyme infused to the patients.

### DESCRIPTION OF THE FIGURES

FIGS. 1A-1E depict the crystal structure of the human α-Gal A (FIGS. 1A-1C[1]). The enzyme monomer (FIG. 1A) has a large amino-terminal catalytic domain (Domain 1) and a small carboxyl-terminal domain (Domain 2) and is present as a head-to-tail homodimer (FIGS. 1B-1C). Active site overlay (FIG. 1D; Stokes, unpublished) of the human (green) and yeast (cyan) enzymes shows the active site mutation Y207W (red and blue). In the dimer interface 30 residues from each monomer contribute to the interface. At the interface of the two monomers (FIG. 1E; Stokes, unpublished) residues Phe 273 (F273 in the linker joining alpha helix 7 to beta sheet 7) and Trp 277 (W277 in alpha helix 7) in Chains A and B make critical contacts. We mutated these (F273C and W277C) along with N278C (Goal 2, MODIP) to Cys residues intended to form disulfide linkages between the monomers. (FIGS. 1A-1C, [1]; FIGS. 1D-1E, Stokes, unpublished).

FIG. 2 depicts the active site of the human a -galactosidase A bound to substrate. Active site residues W47, D92, D93, Y134, C142, K168, D170, C172, E203, Y207, R227, and D231 are shown.

FIG. 3 depicts a colony screening assay for active site mutants expressed in P. pastoris. This qualitative assay detects alpha -galactosidase A activity in vivo using X-α-gal as a substrate in agar media. The D170C mutation is a control indicating that changes in D170 active site residue inactivates the enzyme.

FIG. 4 depicts a Lineweaver-Burke plot of kinetic parameters of WT alpha-galactosidase A on the synthetic substrate, 4-methylumbelliferyl-α-D-galactopyranoside (MUG).

FIG. 5 depicts a Lineweaver-Burke plot of kinetic parameters of Y207W mutant alpha-galactosidase A on the synthetic substrate, 4-methylumbelliferyl-α-D-galactopyranoside (MUG).

FIG. 6 depicts a SDS PAGE gel of the protein content at the different stages of purification of Y207W mutant alpha -galactosidase A. 70.1—Shake flask Supernatant; 70.2—Diafiltration; 70.6—Dialyzed DEAE pool; and 70.10—Concentrated SP pool.

FIG. 7 depicts a Lineweaver-Burke plot of kinetic parameters of E203C mutant alpha-galactosidase A on the synthetic substrate, 4-methylumbelliferyl-α-D-galactopyranoside (MUG).

FIG. 8 depicts a Lineweaver-Burke plot of kinetic parameters of W277C mutant alpha-galactosidase A on the synthetic substrate, 4-methylumbelliferyl-α-D-galactopyranoside (MUG).

### DETAILED DESCRIPTION

The invention provides modified alpha -galactosidase A polypeptides having improved catalytic properties and/or improved dimer stability.

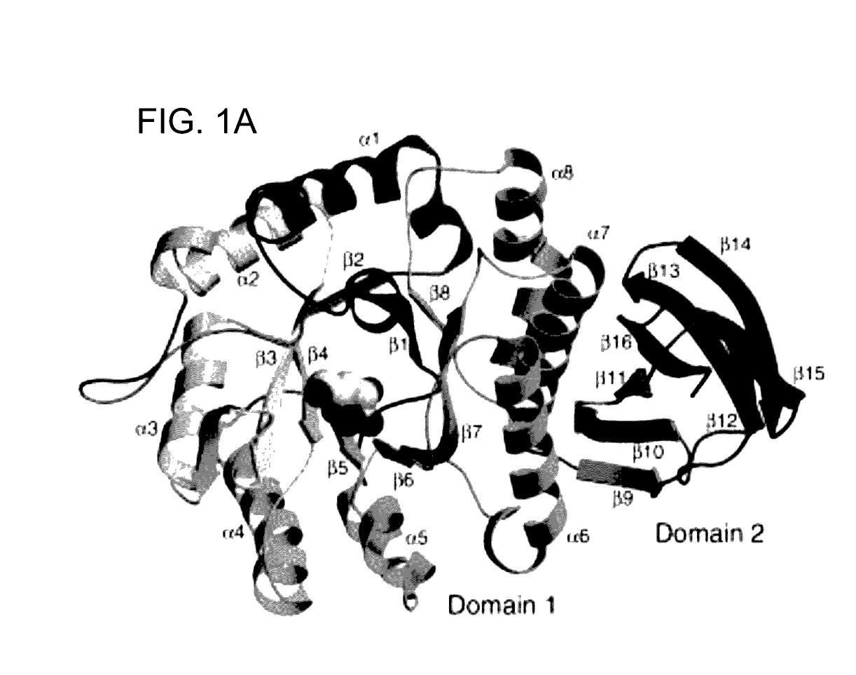

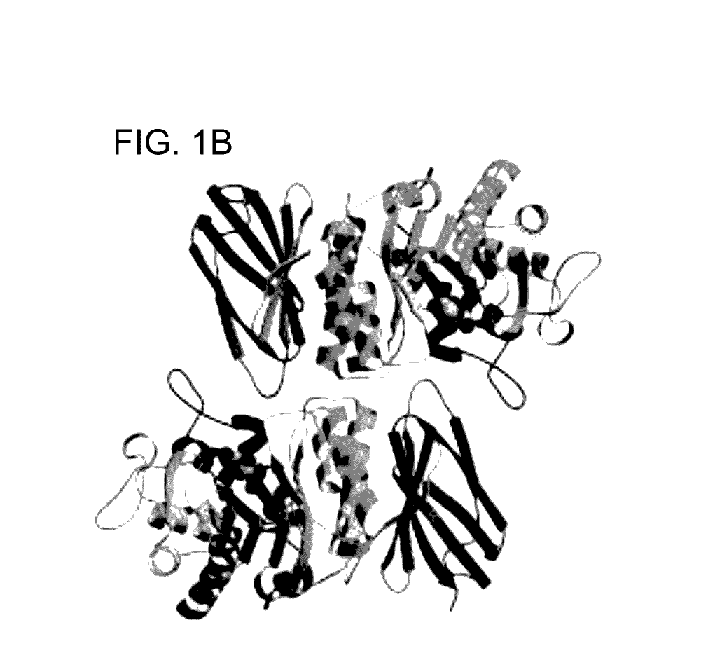

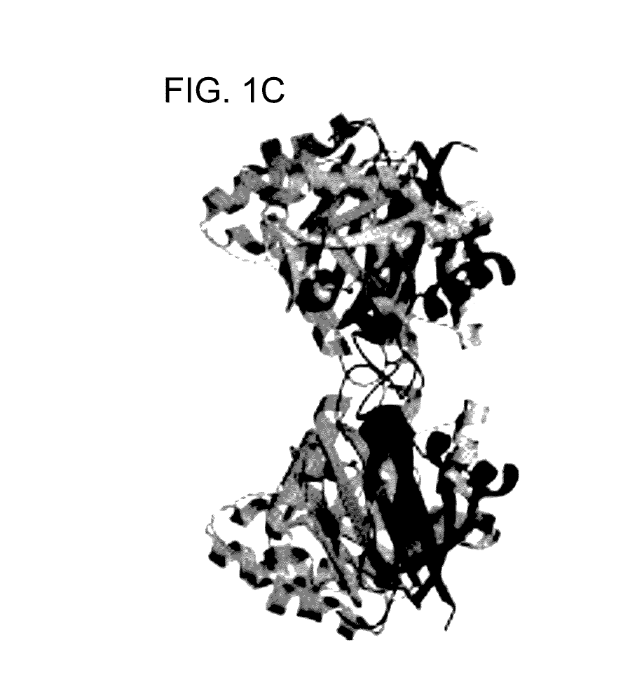

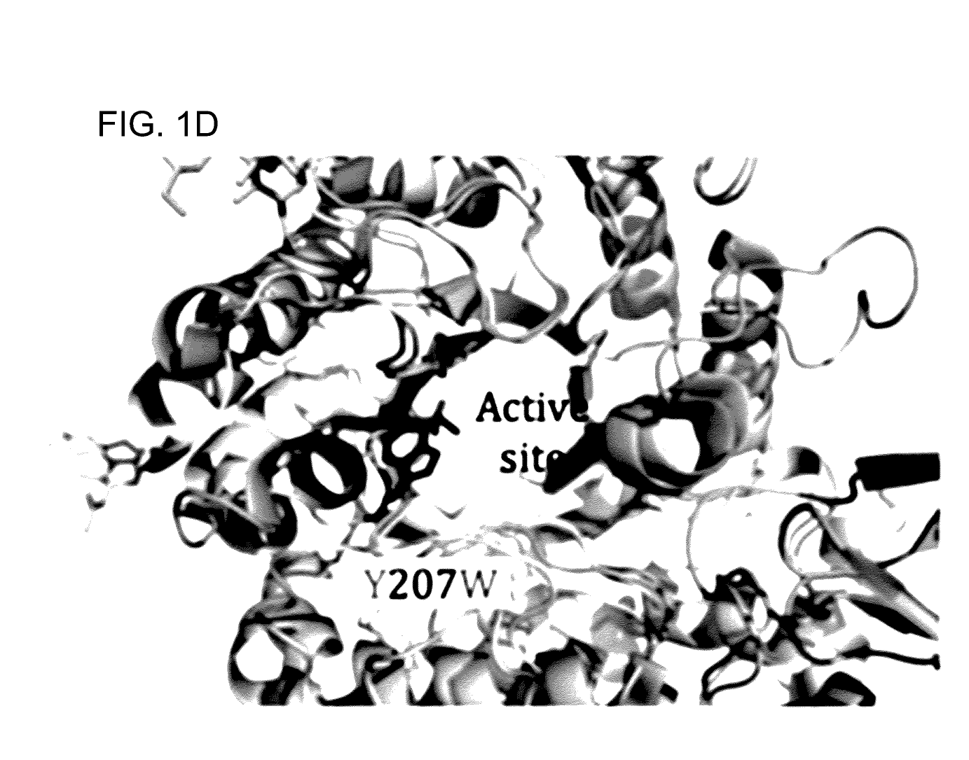

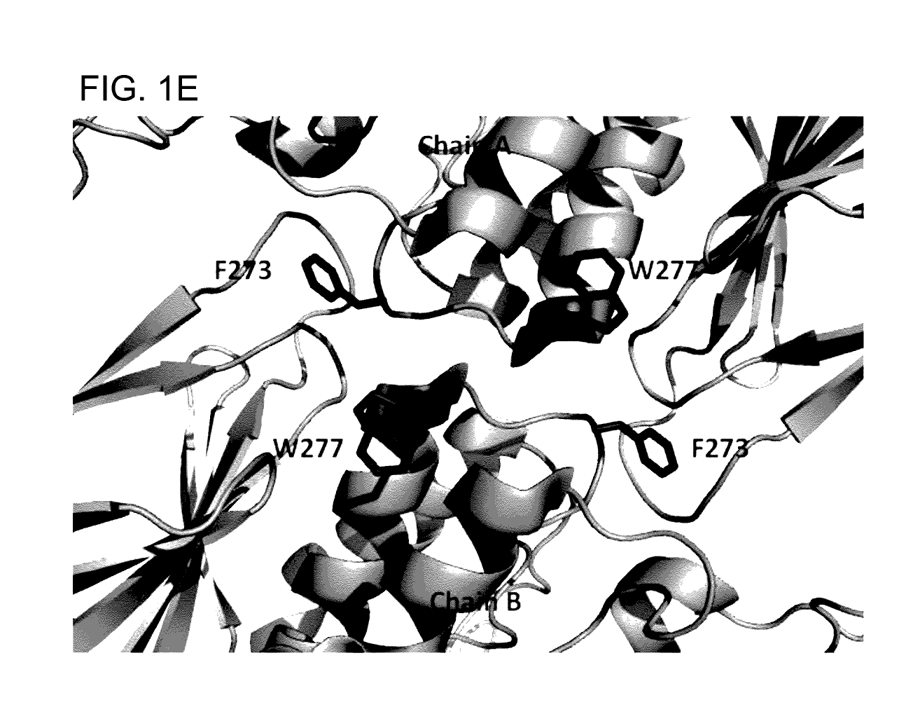

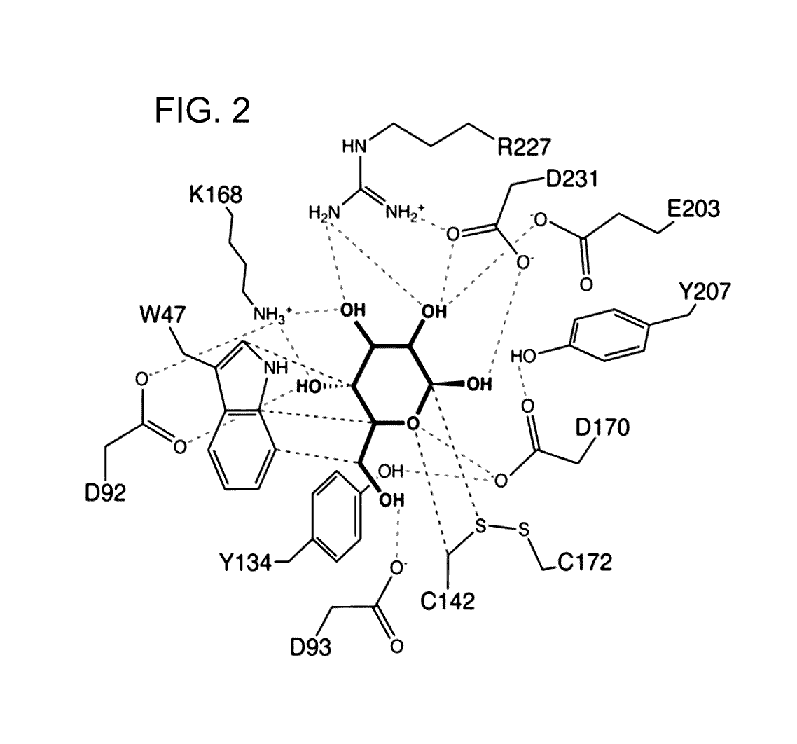

The human alpha -galactosidase A (accession no. NP 000160) is the product of the GLA gene that encodes a precursor form of 429 amino acids (FIG. 1). A 31 amino acid signal peptide is removed during biosynthesis by a signal peptidase to generate a mature form of the enzyme composed of 398 amino acids. There are five disulfide bonds in the enzyme between cysteine residues at positions 52-94, 56-63, 142-172, 202-223 and 378-382 with the 142-172 disulfide bond contributing to the catalytic site of alpha -galactosidase A. The enzyme is a homodimer, and each monomer is includes two domains, a (β/α) 8 domain containing the active site and a C-terminal domain containing eight antiparallel β strands on two sheets in a β sandwich (FIGS. 1A-1D). After removal of the 31-residue signal sequence, the first domain extends from residues 32 to 330 and contains the active site formed by the C-terminal ends of the β strands at the center of a barrel, a typical location for the active site in (β/α)8 domains. The second domain, includes residues 331-429, packs against the first with an extensive interface, burying 2500 Å² of surface area within one monomer. At the dimer interface 30 residues from each monomer contribute to the interface from loops β1-α1, β-α6, β7-α7, β8-α8, β11-β12, and β15-β16.

The enzyme produced in humans and eukaryotic expression vectors is isolated as a homodimer of about 90 kDa with variable amounts of N-linked carbohydrate that includes about 15% of the molecular weight. The most important residue in the dimer interface, F273, has 130 Å²surface area buried per monomer upon formation of the dimer. This residue alone (out of the 30 in the dimer interface) accounts for 12% of the buried surface area in the interface. This residue is a Phe or Tyr in most animal α-galactosidases and α-acetyl-galactosidases, while in plant α-galactosidases, the equivalent residue is a Gly. Thus, this residue predicts the dimerization state of the enzyme in different species: Phe or Tyr indicates the enzyme is a dimer, while Gly indicates the enzyme remains a monomer.

As used herein, the terms “peptide,” “polypeptide,” and “protein” are used interchangeably, and refer to a compound having amino acid residues covalently linked by peptide bonds. A protein or peptide must contain at least two amino acids, and no limitation is placed on the maximum number of amino acids that can include a protein's or peptide's sequence. Polypeptides include any peptide or protein having two or more amino acids joined to each other by peptide bonds. As used herein, the term refers to both short chains, which also commonly are referred to in the art as peptides, oligopeptides, and oligomers, for example, and to longer chains, which generally are referred to in the art as proteins, of which there are many types. “Polypeptides” include, for example, biologically active fragments, substantially homologous polypeptides, functional fragments, functional homologues, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

In one embodiment, the invention provides a modified alpha-galactosidase A polypeptide including SEQ ID NO. 1 having at least one of the following mutations E203C/N, Y207R/W, Y134W, and R227W.

As used herein, residue number corresponds to the position in human alpha galactosidase A, SEQ ID NO. 1.

Generation of modified alpha-galactosidase A polypeptides may be accomplished by any method known in the art. Expression and purification of recombinant alpha-galactosidase A may be accomplished by any known method. For example, generation of mutants, along with protein expression and purification of recombinant proteins are described in Molecular Cloning: A Laboratory Manual (Fourth Edition), Green and Russell ISBN-10: 1936113422, Cold Spring Harbor Laboratory Press; 4th edition, the contents of which are incorporated herein by reference.

In one embodiment, the invention provides a modified alpha-galactosidase A polypeptide of SEQ ID NO. 1 having at least one of the following mutations E203C and Y207W.

In one embodiment, the invention provides a modified alpha-galactosidase A polypeptide of SEQ ID NO. 1 having the Y207W mutation.

In one embodiment, the invention provides a functional homolog or functional fragment of any of the modified alpha-galactosidase A polypeptides disclosed herein.

As used herein, “functional fragment” refers to a polypeptide fragment of the alpha-galactosidase A polypeptide or the nucleic acid sequence encoding thereof, that is capable of providing the same activity as the full length polypeptide and having the subject mutation(s).

As used herein, a “functional homologue” refers to a polypeptide sequence or the nucleic acid sequence encoding for such a protein that is highly homologous to or has a high identity with the alpha-galactosidase A described herein and contains the subject mutation(s). For example, certain amino acid residues can conventionally be replaced by others of comparable nature, e.g. a basic residue by another basic residue, an acidic residue by another acidic residue, a hydrophobic residue by another hydrophobic residue, and so on. Examples of hydrophobic amino acids are valine, leucine and isoleucine. Phenylalanine, tyrosine and tryptophan are examples of amino acids with an aromatic side chain and cysteine as well as methionine are examples of amino acids with sulphur-containing side chains. Serine and threonine contain aliphatic hydroxyl groups and are considered to be hydrophilic. Aspartic acid and glutamic acid are examples of amino acids with an acidic side chain.

In summary, the term “functional homologue” includes variants of the alpha-galactosidase A protein in which amino acids have been inserted, replaced or deleted and which maintain the effects described herein, including catalytic activity and dimeric stability.

Preferred variants are variants which only contain conventional amino acid replacements as described above. Also included in the term “functional homologue” are homologous sequences. Preferably, such a homologue has more than 80% identity on the amino acid sequence level. More preferably, the amino acid has an identity of at least 85% or at least 90%. Even more preferred are sequences that have an identity of at least 91%, at least 92%, at least 93%, at least 94% or at least 95%. Most preferred are sequences that have an identity of 96, 97, 98 or 99% with SEQ ID NO. 1 along with at least one of the mutations described herein, and optionally along with one or more of the conserved residues described herein. Homologous proteins according to the invention have a higher degree of identity with the SEQ ID NO. 1 as the sequences aligned with alpha-galactosidase A enzymes from Mus musculus (NP 038491), Ruminococcus gnavus (WP_004844583.1), Saccharopolyspora erythraea (AAC99325), Saccharomyces cerevisiae (P41947), Clostridium josuil (BAB83765), and Phanerochaete chrysosporium (AAG24511).

In one embodiment, the functional homolog or function fragment of alpha-galactosidase A preferably includes one or more of the following conserved amino acid residuesW47, D92, D93, C142, K168, D170, C172, and D231.

In one embodiment, the invention provides a modified alpha-galactosidase A polypeptide of SEQ ID NO. 1 having at least one of the following mutations E48C, L275C, R301C, H302C, R49C, S304C, G361C, P362C, N408C, P409C, D234C, S235C, N272C, F273C, G274C, S276C, W277C, E358C, I359C, and G360C.

In one embodiment, the invention provides a modified alpha-galactosidase A polypeptide of SEQ ID NO. 1 having at least of the following mutations E48C, L275C, R301C, and H302C.

In one embodiment, the invention provides a modified alpha-galactosidase A polypeptide of SEQ ID NO. 1 having at least one mutation is selected from R49C, S304C, G361C, P362C, N408C, and P409C.

In one embodiment, the invention provides a modified alpha-galactosidase A polypeptide of SEQ ID NO. 1 having at least one mutation is selected from D234C, S235C, N272C, F273C, G274C, S276C, W277C, E358C, I359C, and G360C.

In one embodiment, the invention provides a modified alpha-galactosidase A polypeptide of SEQ ID NO. 1 having at least one mutation selected from F273C and W277C.

In another embodiment, the invention provides a modified alpha-galactosidase A polypeptide of SEQ ID NO. 1, having an E48C mutation; and at least of the following mutations L275C, R301C, H302C, R49C, S304C, G361C, P362C, N408C, P409C, D234C, S235C, N272C, F273C, G274C, S276C, W277C, E358C, I359C, and G360C.

In one embodiment, the invention provides a modified alpha-galactosidase A polypeptide of SEQ ID NO. 1, having an L275C mutation; and at least one of the following mutations E48C, R301C, H302C, R49C, S304C, G361C, P362C, N408C, P409C, D234C, S235C, N272C, F273C, G274C, S276C, W277C, E358C, I359C, and G360C.

In one embodiment, the invention provides a modified alpha-galactosidase A polypeptide of SEQ ID NO. 1, having an R301C mutation; and at least of the following mutations E48C, L275C, H302C, R49C, S304C, G361C, P362C, N408C, P409C, D234C, S235C, N272C, F273C, G274C, S276C, W277C, E358C, I359C, and G360C.

In one embodiment, the invention provides a modified alpha-galactosidase A polypeptide of SEQ ID NO. 1, having an H302C mutation; and of the following mutations E48C, L275C, R301C, R49C, S304C, G361C, P362C, N408C, P409C, D234C, S235C, N272C, F273C, G274C, S276C, W277C, E358C, I359C, and G360C.

In one embodiment, the invention provides a modified alpha-galactosidase A polypeptide of SEQ ID NO. 1, having an E48C mutation; and at least of the following mutations R49C, L275C, R301C, H302C, S304C, G361C, P362C, N408C, P409C, D234C, S235C, N272C, F273C, G274C, S276C, W277C, E358C, I359C, and G360C.

In one embodiment, the invention provides a modified alpha-galactosidase A polypeptide of SEQ ID NO. 1, having an S304C mutation; and at least one of the following mutations E48C, L275C, R301C, H302C, R49C, G361C, P362C, N408C, P409C, D234C, S235C, N272C, F273C, G274C, S276C, W277C, E358C, I359C, and G360C.

In one embodiment, the invention provides a modified alpha-galactosidase A polypeptide of SEQ ID NO. 1, having an G361C mutation; and at least one of the following mutations E48C, L275C, R301C, H302C, R49C, S304C, P362C, N408C, P409C, D234C, S235C, N272C, F273C, G274C, S276C, W277C, E358C, I359C, and G360C.

In one embodiment, the invention provides a modified alpha-galactosidase A polypeptide of SEQ ID NO. 1, having an P362C mutation; and at least one of the following mutations E48C, L275C, R301C, H302C, R49C, S304C, G361C, N408C, P409C, D234C, S235C, N272C, F273C, G274C, S276C, W277C, E358C, I359C, and G360C.

In one embodiment, the invention provides a modified alpha-galactosidase A polypeptide of SEQ ID NO. 1, having an N408C mutation; and at least one of the following mutations E48C, L275C, R301C, H302C, R49C, S304C, G361C, P362C, P409C, D234C, S235C, N272C, F273C, G274C, S276C, W277C, E358C, I359C, and G360C.

In one embodiment, the invention provides a modified alpha-galactosidase A polypeptide of SEQ ID NO. 1, having an P409C mutation; and at least one of the following mutations E48C, L275C, R301C, H302C, R49C, S304C, G361C, P362C, N408C, D234C, S235C, N272C, F273C, G274C, S276C, W277C, E358C, I359C, and G360C.

In one embodiment, the invention provides a modified alpha-galactosidase A polypeptide of SEQ ID NO. 1, having an D234C mutation; and at least one of the following mutations E48C, L275C, R301C, H302C, R49C, S304C, G361C, P362C, N408C, P409C, S235C, N272C, F273C, G274C, S276C, W277C, E358C, I359C, and G360C.

In one embodiment, the invention provides a modified alpha-galactosidase A polypeptide of SEQ ID NO. 1, having an S235C mutation; and at least one of the following mutations E48C, L275C, R301C, H302C, R49C, S304C, G361C, P362C, N408C, P409C, D234C, N272C, F273C, G274C, S276C, W277C, E358C, I359C, and G360C.

In one embodiment, the invention provides a modified alpha-galactosidase A polypeptide of SEQ ID NO. 1, having an N272C mutation; and at least one of the following mutations E48C, L275C, R301C, H302C, R49C, S304C, G361C, P362C, N408C, P409C, D234C, S235C, F273C, G274C, S276C, W277C, E358C, I359C, and G360C.

In one embodiment, the invention provides a modified alpha-galactosidase A polypeptide of SEQ ID NO. 1, having an F273C mutation; and at least one of the following mutations E48C, L275C, R301C, H302C, R49C, S304C, G361C, P362C, N408C, P409C, D234C, S235C, N272C, G274C, S276C, W277C, E358C, I359C, and G360C.

In one embodiment, the invention provides a modified alpha-galactosidase A polypeptide of SEQ ID NO. 1, having an G274C mutation; and at least of the following mutations E48C, L275C, R301C, H302C, R49C, S304C, G361C, P362C, N408C, P409C, D234C, S235C, N272C, F273C, S276C, W277C, E358C, I359C, and G360C.

In one embodiment, the invention provides a modified alpha-galactosidase A polypeptide of SEQ ID NO. 1, having an S276C mutation; and at least one of the following mutations E48C, L275C, R301C, H302C, R49C, S304C, G361C, P362C, N408C, P409C, D234C, S235C, N272C, F273C, G274C, W277C, E358C, I359C, and G360C.

In one embodiment, the invention provides a modified alpha-galactosidase A polypeptide of SEQ ID NO. 1, having an W277C mutation; and at least one of the following mutations E48C, L275C, R301C, H302C, R49C, S304C, G361C, P362C, N408C, P409C, D234C, S235C, N272C, F273C, G274C, S276C, E358C, I359C, and G360C.

In one embodiment, the invention provides a modified alpha-galactosidase A polypeptide of SEQ ID NO. 1, having an E358C mutation; and at least one of the following mutations E48C, L275C, R301C, H302C, R49C, S304C, G361C, P362C, N408C, P409C, D234C, S235C, N272C, F273C, G274C, S276C, W277C, I359C, and G360C.

In one embodiment, the invention provides a modified alpha-galactosidase A polypeptide of SEQ ID NO. 1, having an I359C mutation; and at least of the following mutations E48C, L275C, R301C, H302C, R49C, S304C, G361C, P362C, N408C, P409C, D234C, S235C, N272C, F273C, G274C, S276C, W277C, E358C, and G360C.

In one embodiment, the invention provides a modified alpha-galactosidase A polypeptide of SEQ ID NO. 1, having an G360C mutation; and at least one of the following mutations E48C, L275C, R301C, H302C, R49C, S304C, G361C, P362C, N408C, P409C, D234C, S235C, N272C, F273C, G274C, S276C, W277C, E358C, and I359C.

In another embodiment, the invention provides a modified alpha-galactosidase A polypeptide of SEQ ID NO. 1 having at least one of the following mutations E48C, L275C, R301C, H302C, S304C, G361C, P362C, N408C, P409C, D234C, N272C, F273C, G274C, S276C, W277C, E358C, and I359C.

In another embodiment, the invention provides a stabilized alpha-galactosidase A polypeptide of SEQ ID NO. 1 having at least one of the following mutations E48C, L275C, R301C, H302C, R49C, S304C, G361C, P362C, N408C, P409C, D234C, S235C, N272C, F273C, G274C, S276C, W277C, E358C, I359C, and G360C.

In one embodiment, the stabilized alpha-galactosidase A has an increased half life in physiological conditions. The half life is increased by at least 5%, at least 10%, at least 15%, at least 25%, at least 50%, as compared to the corresponding half life of the WT alpha-galactosidase A polypeptide.

In one embodiment, the stabilized alpha-galactosidase A has at least 5%, at least 10%, at least 15%, at least 25%, at least 50% increased thermostability as compared to the corresponding thermostability of the WT alpha-galactosidase A polypeptide.

In one embodiment, the invention provides a stabilized alpha-galactosidase A polypeptide dimer, wherein each monomer of the dimer includes a polypeptide of SEQ ID NO. 1 and each polypeptide independently having at least one of the following mutations E48C, L275C, R301C, H302C, R49C, S304C, G361C, P362C, N408C, P409C, D234C, S235C, N272C, F273C, G274C, S276C, W277C, E358C, I359C, and G360C.

In one embodiment, the present invention provides a stabilized alpha-galactosidase A polypeptide of SEQ ID NO. 1 having at least one mutation from N272C, F273C, G274C, S276C, and W277C.

In one embodiment, the present invention provides a stabilized alpha-galactosidase A polypeptide of SEQ ID NO. 1 having the W277C mutation.

Without wishing to be bound by theory, it is believed that the stabilized alpha-galactosidase A polypeptide or alpha-galactosidase A dimer disclosed herein provides increased resistance to thermal, proteolytic stress, or lysosomal stress, all of which is believed to lead to increased physiological half-life.

In one embodiment, the stabilized alpha-galactosidase A dimer has an increased half life in physiological conditions. The half life is increased by at least 5%, at least 10%, at least 15%, at least 25%, at least 50%, as compared to the corresponding half life of the WT alpha-galactosidase A enzyme.

In one embodiment, the stabilized alpha-galactosidase A dimer has at least 5%, at least 10%, at least 15%, at least 25%, at least 50% increased thermostability as compared to the corresponding thermostability of the WT alpha-galactosidase A enzyme.

In another embodiment, the invention provides a stabilized alpha-galactosidase A dimer wherein each monomer of the dimer is a polypeptide as described above and is covalently linked by at least one disulfide bond.

The use of non-natural amino acids to establish polypeptide linkages has also been contemplated. Methods of incorporating non-natural amino acids into polypeptides are well known in the art. In one embodiment, applicants have contemplated the use of O-(haloalkyl)-L-tyrosine in place of the cysteine mutations described above. O-(haloalkyl)-L-tyrosine is known to form a covalent thioether bond with cysteine when positioned in close proximity. In one embodiment, the O-(haloalkyl)-L-tyrosine is O-(3-chloropropyl)-L-tyrosine. In another embodiment, the O-(haloalkyl)-L-tyrosine is O-(iodoalkyl)-L-tyrosone.

In one embodiment, the present invention provides a modified alpha-galactosidase A polypeptide of SEQ ID NO. 1 having at least one mutation selected from E203C/N, Y207R/W, Y134W, and R227W; and at least one mutation selected from E48C, L275C, R301C, H302C, R49C, S304C, G361C, P362C, N408C, P409C, D234C, S235C, N272C, F273C, G274C, S276C, W277C, E358C, I359C, and G360C.

In one embodiment, the present invention provides a modified alpha-galactosidase A polypeptide of SEQ ID NO. 1 having the following mutations Y207W and W277C.

In one embodiment, the present invention provides a modified alpha-galactosidase A polypeptide of SEQ ID NO. 1 having the following mutations Y207W and E203C; and W277C.

In another embodiment, the stabilized alpha-galactosidase A dimer according to described herein is covalently linked to at least one other dimer. Covalent linkage may be accomplished by any method known in the art. In one embodiment, the covalent linkage is a disulfide bond. In yet another embodiment, the covalent linkage is via a PEG linker. In yet another embodiment, the PEG linker is a STAR PEG linker.

In another embodiment, the invention provides a fusion protein of an alpha-galactosidase A polypeptide disclosed herein with a targeting peptide. Targeting peptides are well known in the art. As used herein, a targeting peptide is a peptide that localizes in a particular location in a cell or location within an organism. An example of a targeting peptide includes antibodies.

In another embodiment, the invention provides a fusion protein of an alpha-galactosidase A polypeptide disclosed herein with a solubility peptide. Solubility peptides are well known in the art. As used herein, a solubility peptide is a peptide that increases solubility of the bound peptide or polypeptide.

In another embodiment, the invention provides a fusion protein of a modified alpha-galactosidase A polypeptide disclosed herein with a protease resistance peptide. Protease resistance peptides are well known in the art. As used herein, a protease resistance peptide confers at least some degree of protease resistance to the bound peptide or polypeptide.

The polypeptides of the present invention may be in the form of a monomer or multimer. The multimer may be a dimer or a trimer. The monomeric polypeptides may be linked by any method commonly known in the art. In one embodiment, the polypeptides are linked by amino acid linkages. In yet another embodiment, the polypeptides of the present invention are linked by polyethylene glycol (PEG). In yet another embodiment, the polypeptides of the present invention are linked by crosslinkers. Examples of crosslinkers include zero-length crosslinkers, homobifunctional crosslinkers, or heterbifunctional crosslinkers.

In another embodiment, the invention provides a polynucleotide sequence encoding for any of the polypeptides disclosed herein. SEQ ID NO. 2 provides a DNA sequence that encodes for SEQ ID NO. 1. A person of ordinary skill in the art would be able to modify SEQ ID NO. 2 to arrive at any of the modified alpha-galactosidase A polypeptides disclosed herein. Such methods are commonly known in the art. See Molecular Cloning, Green and Russel, above.

In some embodiments, the polynucleotide sequences disclosed herein may be in a vector. Vector includes expression vectors and cloning vectors.

As used herein, the term “polynucleotide” is defined as a chain of nucleotides. Polynucleotide includes DNA and RNA. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and polymerase chain reaction (PCR), and the like, and by synthetic means.

In another embodiment, the compositions of the present invention may be used to treat patients suffering from Fabry disease. Accordingly, the compositions of the present invention may be in the form of a therapeutic agent.

The therapeutic agent can be provided in any suitable form, generally depending upon the desired administration route. For example, the therapeutic agent can be proved as a salt, a solid, a liquid, a suspension, an aggregate, or a gel.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

In accordance with one embodiment, the delivery system and/or delivery vehicle can be provided in conjunction with a local drug delivery apparatus. A local drug delivery apparatus can be a medical device for implantation into a treatment site of a living organism and can include at least one delivery vehicle and/or a therapeutic agent in a therapeutic dosage releasably affixed to the medical device. A local delivery apparatus can include a material for preventing the delivery vehicle and/or the therapeutic agent from separating from the medical device prior to implantation of the medical device at the treatment site, the material being affixed to the medical device or a component of the delivery system.

A delivery vehicle and/or a therapeutic agent may be affixed to any number of medical devices. For example, the delivery vehicles and systems can be associated with or fixed with pumps, catheters, or implants. A delivery system may be affixed to minimize or substantially eliminate the biological organism's reaction to the introduction of the medical device utilized to treat a separate condition. For example, stents, catheters, implants, balloons, self-expandable or nor, degradable or not, can be utilized in conjunction with the delivery system.

The components of the delivery system can be administered in vivo by use of a pharmaceutically acceptable carrier in the form of a composition. By “pharmaceutically acceptable” is meant a material that is not biologically or otherwise undesirable, i.e., the material may be administered to a subject, along with the components of the delivery system, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of pharmaceutically-acceptable carriers include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the delivery system, which matrices are in the form of shaped articles, e.g., films. It will be apparent to those persons skilled in the art that certain carriers may be more preferable depending upon, for instance, the route of administration and concentration of composition being administered.

Pharmaceutical compositions for use in conjunction with the delivery system may include carriers, thickeners, diluents, buffers, preservatives, surface active agents and the like in addition to the delivery vehicle and/or the therapeutic agent. Pharmaceutical compositions may also include one or more active ingredients such as antimicrobial agents, anti-inflammatory agents, anesthetics, and the like.

A pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The delivery system can be administered intravenously, intraperitoneally, intramuscularly, subcutaneously, intracavity, or transdermally.

Parenteral administration, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained.

Preparations for parenteral administration can include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral carriers include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like.

Preservatives and other additives may also be present such as, for example, antimicrobials, anti-oxidants, chelating agents, and inert gases and the like.

Compositions for oral administration include powders or granules, suspensions or solutions in water or non-aqueous media, capsules, sachets, or tablets. Thickeners, flavorings, diluents, emulsifiers, dispersing aids or binders may be desirable.

A composition may be administered by use of a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the delivery system may be determined empirically, and making such determinations is within the skill in the art. The dosage ranges for the administration of the delivery system are those large enough to produce the desired effect. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products.

In one embodiment, the present invention therefore, provides a method of treating a subject suffering from Fabry disease by administering an effective amount of the polypeptide disclosed herein to a subject in need thereof.

The terms “effective amount” and “therapeutically effective amount” of a modified alpha-galactosidase A polypeptide as used herein means a sufficient amount of the a modified alpha-galactosidase A polypeptide to provide the desired therapeutic or physiological or effect or outcome. Such, an effect or outcome includes reduction or amelioration of the symptoms of cellular disease. Undesirable effects, e.g. side effects, are sometimes manifested along with the desired therapeutic effect; hence, a practitioner balances the potential benefits against the potential risks in determining what an appropriate “effective amount” is. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation. Generally, the engineered cell or engineered cells is/are given in an amount and under conditions sufficient to reduce proliferation of target cells.

The terms “subject,” “individual,” and “patient” are used interchangeably herein to refer to a mammal. In an embodiment, the mammal is a human. The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

The present disclosure may be better understood with reference to the examples, set forth below. The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

In the specification, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

Throughout this specification, quantities are defined by ranges, and by lower and upper boundaries of ranges. Each lower boundary can be combined with each upper boundary to define a range. The lower and upper boundaries should each be taken as a separate element.

Reference throughout this specification to “one embodiment,” “an embodiment,” “one example,” or “an example” means that a particular feature, structure or characteristic described in connection with the embodiment or example is included in at least one embodiment of the present embodiments. Thus, appearances of the phrases “in one embodiment,” “in an embodiment,” “one example,” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus.

Further, unless expressly stated to the contrary, “or” refers to an inclusive “or” and not to an exclusive “or”. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

In this specification, groups of various parameters containing multiple members are described. Within a group of parameters, each member may be combined with any one or more of the other members to make additional sub-groups. For example, if the members of a group are a, b, c, d, and e, additional sub-groups specifically contemplated include any one, two, three, or four of the members, e.g., a and c; a, d, and e; b, c, d, and e; etc.

While there have been described what are presently believed to be the preferred embodiments of the present invention, those skilled in the art will realize that other and further changes and modifications may be made thereto without departing from the spirit of the invention, and it is intended to claim all such modifications and changes as come within the true scope of the invention.

The present invention is illustrated in further details by the following non-limiting examples.

### EXAMPLES

### Cell Strains and Plasmids.

The P. pastoris host strain X-33 (No. K1740-01), E. coli strains TOP10 (No. C4040-50) and TOP10F′ (No. C665-11), plasmid pPICZaA (No. K1740-01), and TOPO® XL PCR cloning kit (No. K4700-10) were purchased from Invitrogen.

Bioreactor Expression of Recombinant αGal in P. pastoris.

High-cell-density fermentation was carried out as previously described (Chen 2000) with a modified growth medium utilizing non-precipitating sodium hexametaphosphate as a phosphate source (Zhang 2000) and modified for a 7 L Applikon bioreactor. Fermentation medium of 3.5 L (0.93 g/l CaSO4, 18.2 g/l (2504, 14.9 g/l MgSO4.7 H20, 9 g/l (NH4)2SO4, 40.0 g/l glycerol) was autoclaved at 121° C. for 20 min in the vessel. After cooling to room temperature, filter sterilized sodium hexametaphosphate (25 g/l of fermentation basal salt medium dissolved in 500 ml of deionized water) and 0.435% PTM1 trace elements (CuSO4.5 H2O 6.0 g, NaI 0.08 g, MgSO4.H20 3.0 g, Na2MoO4.2 H2O 0.2 g, H3BO3 0.02 g, CoCl2 0.5 g, ZnCl2 20.0 g, FeSO4.7 H2O 65.0 g, biotin 0.2 g, 5.0 ml H2SO4 per liter) were added to complete the fermentation medium. The pH was adjusted to 6.0 using ammonium hydroxide (28%).

Four frozen MGY cultures of 4 ml each were used to inoculate four 100 ml MGY cultures in 1-liter baffled flasks and grown at 250 rpm and 30° C. until the OD600 reached 2 to 6. The cultivation was divided into three phases, the glycerol batch, glycerol-fed batch, and methanol-fed batch. The glycerol batch phase was initiated with 400 ml of inoculum shake-flask culture added to 4 L of the fermentation medium containing 4% glycerol and an initial value of 100% dissolved oxygen until a spike was observed indicating complete consumption of glycerol. Next, the glycerol-fed batch phase was initiated and a 50% w/v glycerol feed rate of 18.15 ml/h/liter initial fermentation volume and maintained until a cell yield of 180 to 220 g/liter wet cells was achieved. At this point the glycerol feed was terminated manually and a methanol-fed batch phase was initiated by starting a 100% methanol feed containing 12 ml PTM1 trace salts per liter. Methanol was initially fed at 3.6 ml/h/liter of initial fermentation volume, then increased to 7.3 ml/h/liter and finally increased to 10.9 ml/h/liter of initial fermentation volume for the remainder of the fermentation. Dissolved oxygen spikes were used during the glycerol-fed batch phase and methanol-fed batch phase and to monitor substrate levels. A dissolved oxygen level of 40%, pH of 6, and temperature of 25° C. were maintained by an ADI 1030 regulator. Sampling was performed at the end of each phase and at lease twice daily and analyzed for cell wet weight and increased αGal activity over time. Cultivation was terminated once a plateau in αGal activity was observed.

Purification of αGal using Double Affinity Chromatography.

Purification was as described (Chen 2000, Yasuda 2004) with minor modifications (below). Bioreactor supernatant was passed through a 0.2 μm hollow fiber filter (Spectrum Labs, No. M22M-300-01N) and subjected to diafiltration using a 50 kDa pore size hollow fiber filter (Spectrum Labs, No. M25S-300-01N) against wash buffer (0.1 M sodium acetate buffer, pH 6.0, 0.1 M NaCl, 1 mM MgCl2, 1 mM CaCl2, 1 mM MnCl2). The resulting supernatant was applied to a Con A Sepharose 4B (GE Healthcare No. 17-0440-01) column, pre-equilibrated with wash buffer, and washed with 5 column volumes of wash buffer. It was observed that near-saturating sugar eluent concentrations do not improve glycoprotein recovery as compared to lower concentrations and that elution phase pauses improve recovery. In accordance with these findings, elution of αGal was carried out using modified elution buffer I (0.5 M methyl-α-d-mannopyranoside, 0.25 M methyl-α-d-glucopyranoside in wash buffer) over 1.5 column volume blocks separated by 12-hour interval soaks. Elution was discontinued when the absorbance at 280 nm and enzyme assays showed negligible presence of protein and αGal activity. No substantial difference in recovered enzyme was observed between purifications carried out with modified elution buffer I versus sugar saturated elution buffer I (data not shown). The Con A pool was subjected to diafiltration using a 50 kDa pore size hollow fiber filter (Spectrum Labs, No. M25S-300-01N) against binding buffer (25 mM citrate-phosphate buffer, pH 4.8 containing 0.1 M NaCl).

The Con A pool was applied to an immobilized-d-galactose gel column (Thio-Gal, Pierce No. 20372) pre-equilibrated with binding buffer. The column was washed with 5 column volumes of binding buffer and αGal was eluted with elution buffer II (25 mM citrate-phosphate buffer, pH 5.5, 0.1 M NaCl, 0.1 M d-galactose) over 1.5 column volume blocks separated by 12 hour soaks. Fractions were assayed for enzyme activity and protein concentration and a peak tube with high specific activity was chosen as the sample to be used in a substrate saturation curve.

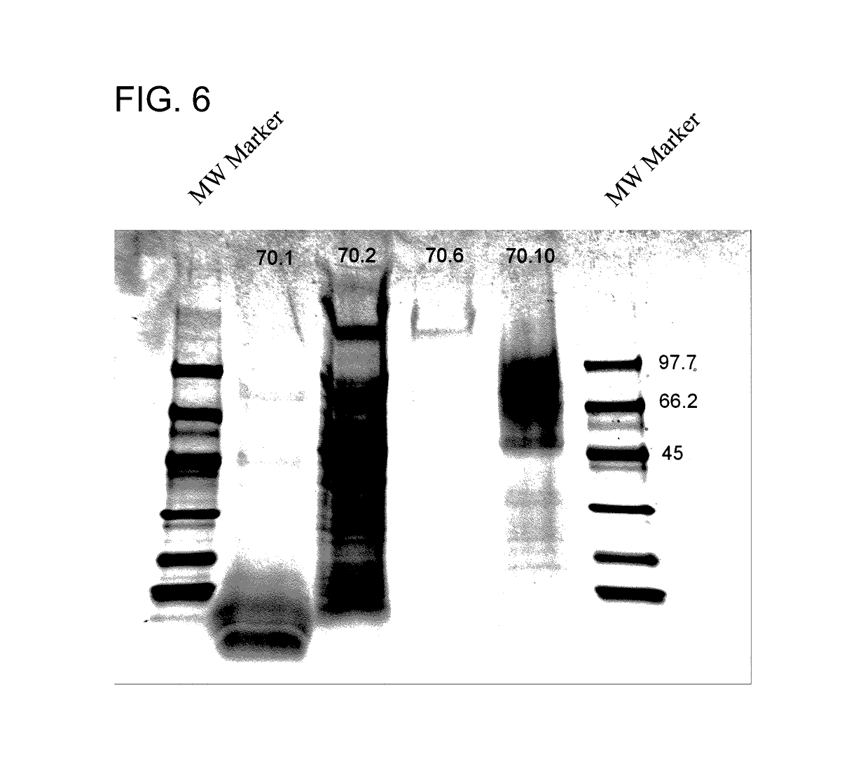

The purification protocol is modified for the purification of the Y207W mutant of alpha-galactosidase A. For this mutant, DEAE (Diethylaminoethyl) and SP (sulphopropyl, strong cation exchange) affinity media are used for purification. See FIG. 6.

### Electrophoresis Analysis

Samples (8 μg) were mixed with an equal volume of reducing sample buffer (Bio-Rad Laemmli sample buffer with 5% β-mercaptoethanol) and heated for 5 minutes at 95° C. before loading on a Mini-Protean TGX Precast Gel 4-20% (w/v) (Bio-Rad No. 456-1094). Bands were visualized by Coomassie blue staining via the modified Fairbanks protocol.

### Western Blot Analysis

Western blot analysis was performed using an anti-αGal polyclonal antibody produced in chicken (Pierce/ThermoSci #PA1-9528) and horseradish peroxidase-conjugated anti-Chicken IgY antibody (Sigma #A9046). After SDS-PAGE (2 μg of samples loaded), the gel was incubated with a nitrocellulose membrane (Whatman, No. 10402594) for 15 minutes at room temperature in Transfer Buffer (48 mM Tris, 39 mM glycine, 20% MeOH, pH 9.2) and the proteins were then transferred to the nitrocellulose membrane using a Bio-Rad Trans Blot SD Semi-Dry Transfer Cell. The membrane was blocked with 8% (w/v) non-fat dried milk in PBST [10 mM Na2HPO4, 1.8 mM KH2PO4, 137 mM NaCl, 2.7 mM KCl and 0.2% Tween 20 (pH 7.4)] at room temperature for 20 minutes. The membrane was then treated with primary antibody diluted in a milk/blot solution [1% (w/v) non-fat dried PBST] for 2 h at room temperature with mild shaking. After rinsing with PBST solution, the membrane was treated for 1 h at room temperature with secondary antibody diluted in the milk/blot solution. Protein bands were visualized on Kodak BioMax XAR film (VWR #lB1651454) with a Konica SRX-101A processor.

### Enzyme and Protein Assays

Activity of αGal was assayed using the synthetic substrate, 4-methylumbelliferyl-α-d-galactopyranoside (MUG) as described (Chen 2000) with modifications to a microtiter plate format (below). Enzyme activity is measured in units/ml where one unit is defined as the amount of enzyme required to convert 1 nmole of MUG to 4-methylumbelliferone in one hour at 37° C. An aliquot of 3 μl was added to 27 μl of enzyme assay buffer (5 mM MUG in 40 mM sodium acetate buffer, pH 4.5). This mixture was incubated at 37° C. and 10 μl aliquots were taken at two time points and added to 290 μl of 0.1 M diethylamine in a microtiter plate to stop the reaction. Typically time points were chosen as 1-4 minutes and values that were proportional to time were considered valid. The fluorescence of each sample was measured at an excitation wavelength of 365 nm and an emission wavelength of 450 nm using a Tecan Infinite F200 microtiter plate reader. A standard curve of 10 μl of 0-0.5 nmol 4-methylumbelliferone dissolved in MeOH in 290 μl of 0.1 M diethylamine was used to quantitate MUG cleavage at specific time intervals. Analysis of the effects of MeOH indicated no effect on the 4-methylumbelliferone standard curve.

For samples containing higher protein concentrations, the BioRad DC Protein Assay (No. 500-0116) with a standard curve of (0.2-1.5) mg/ml was used according to the manufacturer's specifications. For dilute samples of purified αGal, a more sensitive fluorescence-based fluorescamine assay with a standard curve containing lower protein concentrations of (4.0-160) 1 μg/ml was used. Briefly, 150 μl of 0.05 M sodium phosphate buffer and 50 μl of 1.08 mM fluorescamine dissolved in acetone were added to an aliquot of 50 μl of the sample and standards, mixed and incubated for 12 minutes. The fluorescence of each sample was measured at an excitation wavelength of 400 nm and an emission wavelength of 460 nm. Bovine serum albumin (Bio-Rad No. 500-0112) was used as the standard in both assays. Absorbance and fluorescence measurements were conducted on a Tecan Infinite F200 microplate reader using 96-well plates.

### Mass Spectrometry of a Purified Mutant Enzyme

Mass spectrometry is used to analyze the mutant enzyme. SDS-PAGE gel slices are washed, de-stained, reduced using 10 mM dithiothreitol, alkylated using 100 mM iodoacetamide, and digested using trypsin. Peptides are then extracted from the gel two times, dried, and re-suspended in a 5% acetonitrile and 2% formic acid mixture. One third of each sample is loaded onto a C18 PepMap1000 micro-precolumn (300 μm I.D., 5 mm length, 5 μm beads, Thermo Scientific) at a flow-rate of 5 μl/min, and subsequently onto an analytical C18 column (75 μm I.D., 3 μm beads, Nikkyo Technos Co.) at a flow rate of 300 nl/min. The gradient was 40 min long in the range 5 to 45% B (buffer A was 0.1% formic acid in water, and buffer B was 0.1% formic acid in acetonitrile). Eluted peptides are applied by electrospray directly into the LTQ-Orbitrap XL mass spectrometer from Thermo Scientific, operating in a 300 to 1800 m/z mass range. Tandem mass spectrometry was performed by collision induced dissociation using nitrogen as a collision gas. The resulting spectra were analyzed using Mascot and Proteome Discoverer 1.3 (Thermo Scientific) to identify the peptides in the sample.

### Thermo Stability and pH Optimum of WT and Mutant αGal

Purified enzyme samples are diluted in 25 mM citrate-phosphate buffer, pH 5.5, 0.1 M NaCl, 0.01 M D-galactose. Samples of 50 μl were incubated in triplicate at 50° C., 30° C., and 40° C. Aliquots of 3 μl are removed for enzyme assays every 15 minutes for two hours. Samples are assayed in 0.02 M citrate buffer, pH 3.0-pH 6.5, containing 2 mM MUG.

### Characterization of Kinetic Properties

Substrate saturation curves for αGal have been reported using MUG at concentrations up to 2 mM, 5 mM, and 10 mM (in the presence of 0.1% BSA and 0.67% EtOH [24]). We noted that under our experimental conditions MUG is fully soluble at 2 mM, partially soluble at 5 mM, and chemically oversaturated at higher concentrations. Other investigators reported the use of sonication or detergent treatment to increase the solubility of MUG (e.g., [69]) but we avoided this approach in order to avoid potential artifacts due to the use of these techniques. Substrate saturation curves using 2 mM and 5 mM MUG as the highest concentrations were carried out and the kinetic parameters for αGal were calculated separately obtaining similar values. The values reported here (Table 3a) were obtained using a substrate saturation curve of 0.3 to 2 mM MUG since this is the highest concentration that is fully soluble under our experimental conditions. The Km and Vmax values were calculated using Lineweaver-Burk and non-linear regression through the program Sigma-Plot (Systat Software, San Jose, Calif.).

Kinetic parameters were also determined using the colorimetric substrate, para-nitrophenyl-α-d-galactopyranoside (PNPαGal) [70]. Purified enzymes were diluted to approximately 20,000 units/mL as determined by fluorescent MUG assay. These diluted samples were then added at a proportion of 1:9 citrate-phosphate buffer (0.1 M) containing 7-50 mM PNPαGal. Aliquots of 20 μl of the enzymatic reaction were removed at 15 minute intervals to terminate the reaction over the course of an hour and added to 320 μl of borate buffer (pH 9.8) in a microplate [71]. Product formation was monitored by absorbance at 400 nm. Linear reaction velocities were observed for all measurements. A standard curve of 0-150 μM p-nitrophenylate in borate buffer (pH 9.8) [71] was used to quantitate product formation. Km and Vmax parameters were determined through non-linear regression using Sigma-Plot (Systat Software, San Jose, Calif.).

### Protein Structure Analysis

The crystal structure of αGal (PDB 1R47) was viewed and analyzed in PyMOL (Delano Scientific). The MSLDKLL and QMSLKDLL peptides corresponding to the last 7 or 8 C-terminal amino acids of αGal were built in PyRosetta [72] and visualized in PyMOL [73]. Interatomic distances were measured using the PyMOL wizard distance command.

A homology model of the coffee bean α-galactosidase was generated on the Phyre2 server. The primary sequence of coffee bean α-galactosidase (GenBank No. AAA33022.1) was set as the query. The crystal structure of rice α-galactosidase (73% sequence identity to coffee α-galactosidase, PDB# 1UAS) was set as the template. Superposition of the coffee homolog and human crystal structure of αGal (PDB# 1R47) was conducted in PyMOL. Primary sequence alignments were carried out in ClustalOmega (EMBL-EBI).

### In Situ Enzyme Assay.

In situ enzyme assay for colonies of P. pastoris using the artificial substrate X-a-Gal. In situ enzyme assays (12) used nitrocellulose membranes (No. 21850, 0.45-mm pore size, VWR, Plainfield, N.J.) placed on YPDS petri plates with zeocin (100 mg/ml). Colonies that grew on selective media after electroporation were patched onto the membranes using sterile wooden toothpicks (Diamond Brands, Inc., Minneapolis, Minn.). The colonies were grown directly on the membrane overnight at 30° C. and then transferred to the surface of an MM plate for the enzyme induction by methanol. After incubation at 30° C. overnight, the membranes with colonies growing on top of them were placed on Whatman No. 4 filters (No. 1001 125, Whatman, Inc., Clifton, N.J.) saturated with the chromogenic substrate X-α-gal (No. 917591, Boehringer Mannheim, Indianapolis, Ind.) solution (1 mg/ml) in 40 mM sodium acetate, pH 4.5, and incubated at 37° C. Those colonies positive for a-galactosidase A activity were visualized by a change in color from light yellow to blue 1 to 3 h after exposure to the substrate. This assay was adapted from Zhu, A., Monahan, C., Zhang, Z., Hurst, R., Leng, L., and Goldstein, J. (1995) High-level expression and purification of coffee bean α-galactosidase produced in the yeast Pichia pastoris. Arch. Biochem. Biophys. 324, 65-70.

### Example 1

### In situ enzyme assay modified alpha-galactosidase A polypeptide having different combinations of E203C, Y207W, and D170C mutations.

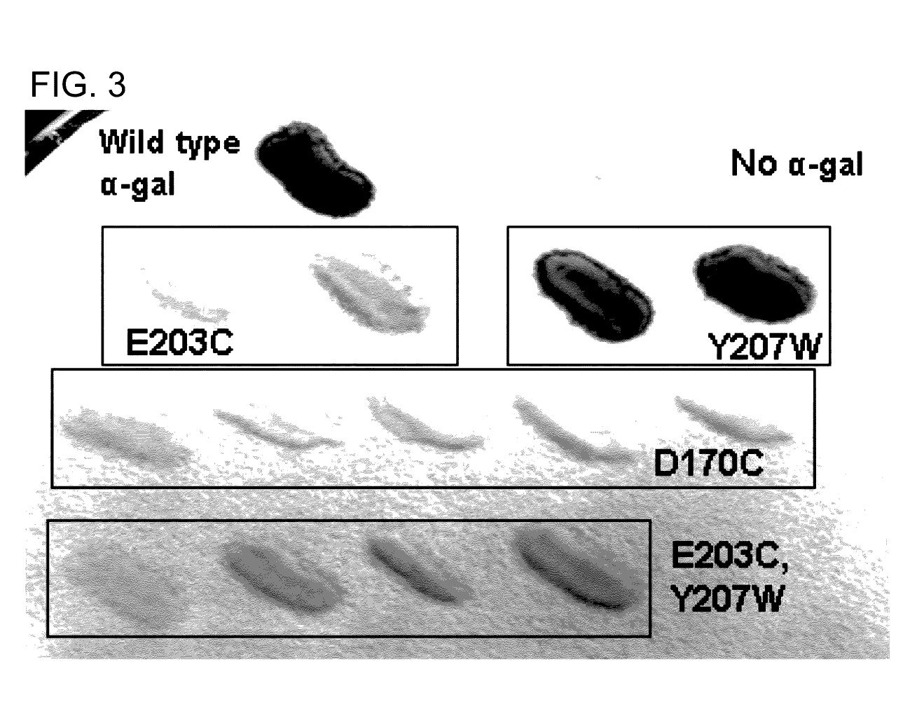

The in situ enzyme assay described above was used to test the following modified alpha galactosidase A polypeptides: WT, E203, Y207W, D170C, and E203C/Y207W. See FIG. 3.

### Example 2

### Kinetic Charactarization of WT Alpha-Galactosidase A Polypeptide

WT alpha-galactosidase A polypeptide was expressed and purified by the methods described above.

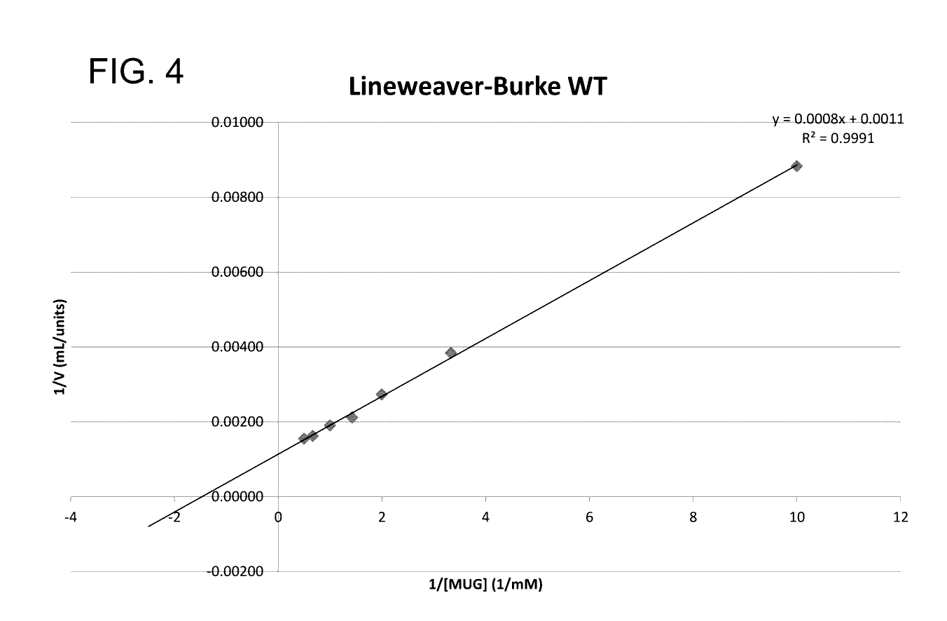

Characterization of kinetic properties where determined as described above. Table 1 provides the various kinetic parameters. See FIG. 4 for the Lineweaver-Burke plot showing the same.

TABLE 1

| | | | | Relative |
|---|---|---|---|---|
| | | | kcat/Km | kcat/Km |
| Km (mM) | Vmax (mmol/hr/mg) | kcat (1/s) | (1/sM) | to WT |
| 0.66 ± | 0.028 ± | 0.69 ± | 1,056 ± | 1 |
| 0.07 | 0.0014 | 0.04 | 83 | |

### Example 3

### Kinetic Charactarization of Modified alpha-Galactosidase A Polypeptide having the W277C Mutation

Modified alpha-galactosidase A polypeptide having the W277C mutation was expressed and purified by the methods described above.

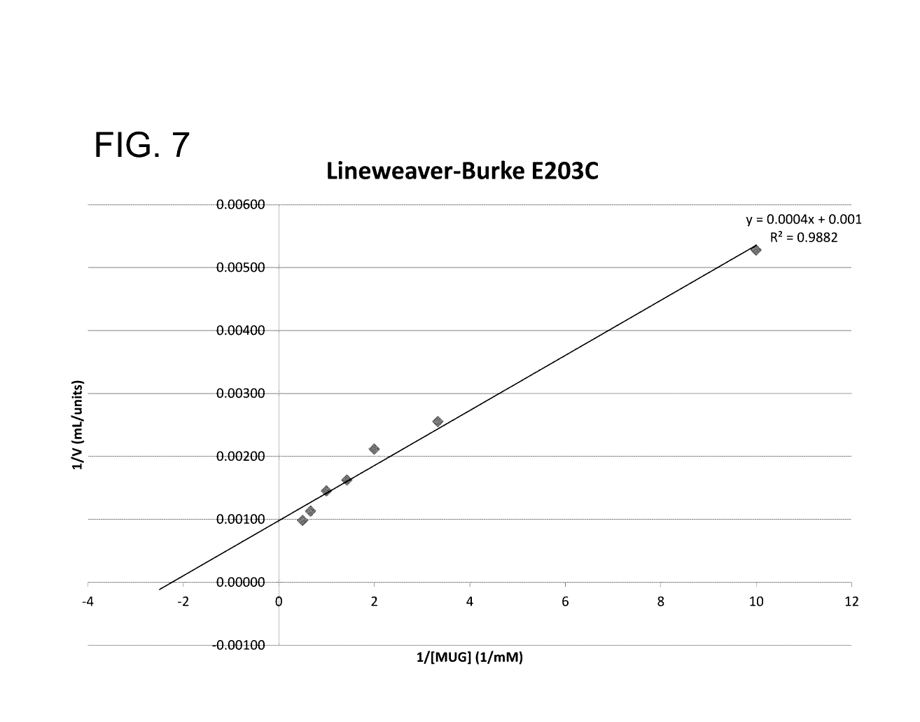

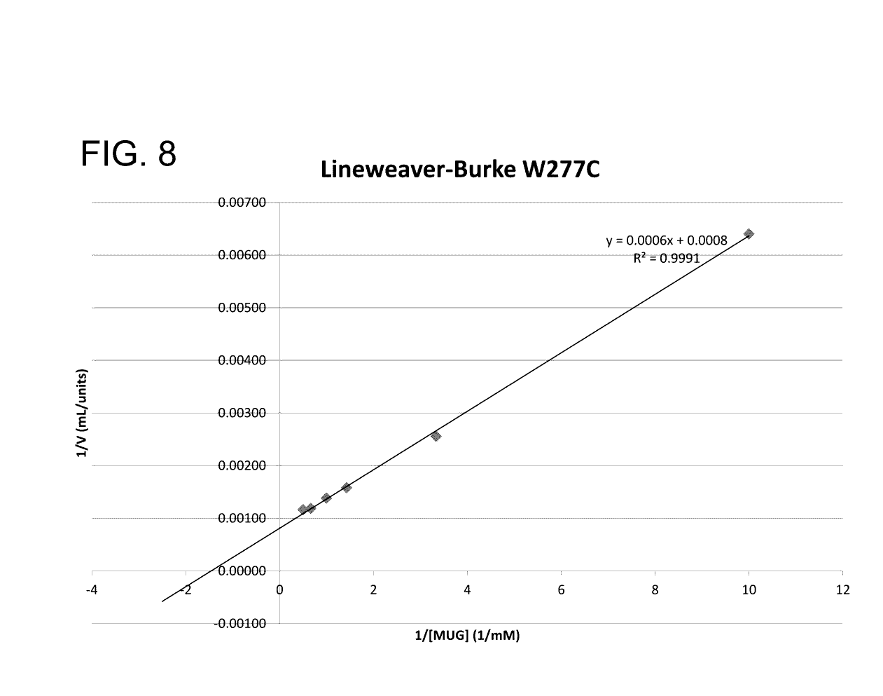

Characterization of kinetic properties where determined as described above. Table 1 provides the various kinetic parameters. See FIG. 7 and FIG. 8 for the Lineweaver-Burke plot showing the same.

TABLE 2

| | | Vmax | | | Relative |
|---|---|---|---|---|---|
| | | (mmol/ | kcat | kcat/Km | kcat/Km |
| MUTATION | Km (mM) | hr/mg) | (1/s) | (1/sM) | to WT |
| W277C | 0.69 ± | 0.019 ± | 0.48 ± | 702 ± | 0.665 |
| | 0.11 | 0.0013 | 0.03 | 76 | |

### Example 4

### Isolation and Kinetic Charactarization of Modified Alpha-Galactosidase A Polypeptide having the Y207W Mutation or the E203C Mutation

Modified alpha-galactosidase A polypeptide having the Y207W mutation was expressed as described above. A purification scheme using DEAE (Diethylaminoethyl) and SP (sulphopropyl, strong cation exchange) affinity media were used for purification. See FIG. 6.

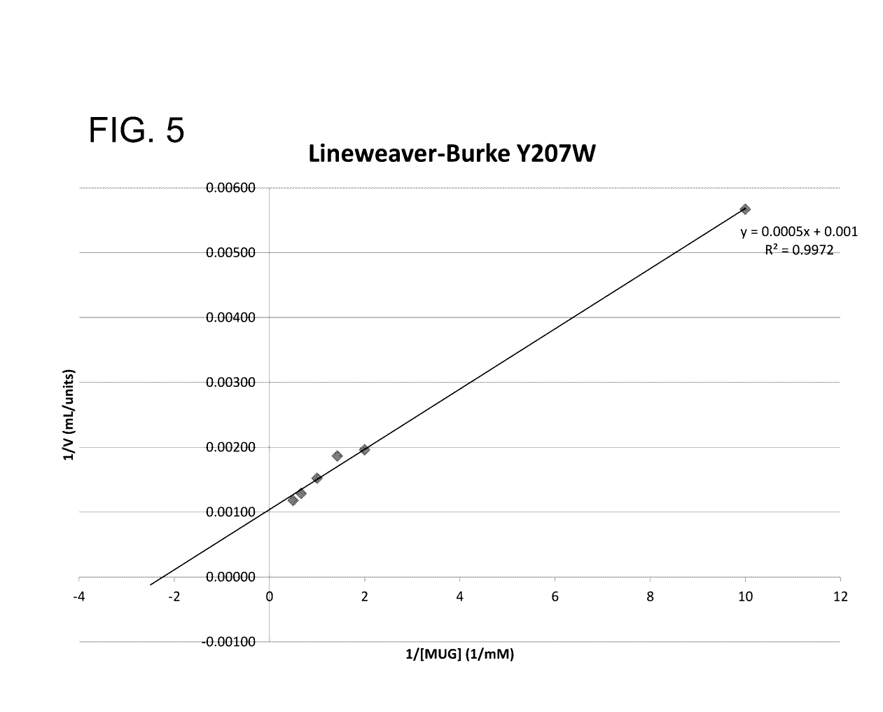

Characterization of kinetic properties where determined as described above. Table 2 provides the various kinetic parameters. See FIG. 5 for the Lineweaver-Burke plot showing the same.

TABLE 3

| | | Vmax | | | Relative |
|---|---|---|---|---|---|
| | | (mmol/ | kcat | kcat/Km | kcat/Km |
| MUTATION | Km (mM) | hr/mg) | (1/s) | (1/sM) | to WT |
| Y207W | 0.47 ± | 0.071 ± | 1.79 ± | 3,869 ± | 3.664 |
| | 0.06 | 0 | 0 | 270 | |
| E203C | 0.48 ± | 0.012 ± | 0.29 ± | 616 ± | 0.583 |
| | 0.03 | 0.0007 | 0.02 | 82 | |

### Example 5

### Isolation and Kinetic Charactarization of Modified Alpha-Galactosidase A Polypeptide having the Y203N Mutation and Y134W Mutation; and Alpha-Galactosidase A Polypeptide having the Y207R Mutation and R227W Mutation.

### Example 6

### Isolation and Kinetic Charactarization of Modified Alpha-Galactosidase A Polypeptide having the Y277C Mutation

Alpha-galactosidase A polypeptides having the Y277C mutation are expressed and purified by methods described above.

Thermostability and pH optimum testing is done as described above.

### Example 7

### Isolation and Kinetic Charactarization of Stabilized Alpha-Galactosidase A Polypeptide Dimers

Alpha-galactosidase A polypeptides having the Y207W mutation and W277C mutation are expressed and purified by methods described above.

Characterization of kinetic properties are determined as described above. Thermostability and pH optimum testing is done as described above.

### REFERENCES

1. Chen Y, Jin M, Egborge T, Coppola G, Andre J, et al. (2000) Expression and characterization of glycosylated and catalytically active recombinant human alpha-galactosidase A produced in Pichia pastoris. Protein Expr Purif 20: 472-484.
2. Chen Y, Jin M, Goodrich L, Smith G, Coppola G, et al. (2000) Purification and characterization of human alpha-galactosidase A expressed in insect cells using a baculovirus vector. Protein Expr Purif 20: 228-236.
3. Coppola G, Yan Y, Hantzopoulos P, Segura E, Stroh J G, et al. (1994) Characterization of glycosylated and catalytically active recombinant human alpha-galactosidase A using a baculovirus vector. Gene 144: 197-203.
4. Guce A T, Clark N E, Salgado E N, Ivanen D R, Kulminskaya A A, et al. (2010) Catalytic mechanism of human alpha-galactosidase. J Biol Chem 285: 3625-3632.
5. Meghdari M, Gao N, Abdullahi A, Stokes E, Calhoun D H (2015) Carboxyl-Terminal Truncations Alter the Activity of the Human alpha-Galactosidase A. PLoS One 10: e0118341.
6. Pluddemann A, Neyen C, Gordon S (2007) Macrophage scavenger receptors and host-derived ligands. Methods 43: 207-217.
7. Garman S C, Garboczi D N (2004) The molecular defect leading to Fabry disease: structure of human alpha-galactosidase. J Mol Biol 337: 319-335.
8. Stephen S L, Freestone K, Dunn S, Twigg M W, Homer-Vanniasinkam S, et al. (2010) Scavenger receptors and their potential as therapeutic targets in the treatment of cardiovascular disease. Int J Hypertens 2010: 646929.
9. Desnick-R J I-Y, Eng-C M (1995) a-Galactosidase A Defiency: Fabry Disease. In: Scriver-CB S-A, Sly-W S, Valle, D, editor. The Metabolic Basis of Inherited Disease. New York: McGraw-Hill. pp. 2741-2784eng-cm.
10. Calhoun D H, Bishop D F, Bernstein H S, Quinn M, Hantzopoulos P, et al. (1985) Fabry disease: isolation of a cDNA clone encoding human alpha-galactosidase A. Proc Natl Acad Sci USA 82: 7364-7368.
11. Quinn M, Hantzopoulos P, Fidanza V, Calhoun D H (1987) A genomic clone containing the promoter for the gene encoding the human lysosomal enzyme, alpha-galactosidase A. Gene 58:177-188.
12. Hantzopoulos P A, Calhoun D H (1987) Expression of the human alpha-galactosidase A in Escherichia coli K-12. Gene 57: 159-169.
13. Desnick R J, Schuchman E H (2002) Enzyme replacement and enhancement therapies: lessons from lysosomal disorders. Nat Rev Genet 3: 954-966.
14. Brady R O, Schiffmann, R. (2007) Possible future therapies for Fabry disease. In: Mehta A, Beck, M., Sunder-Plassmann, editor. Fabry Disease 2007 Book. pp. Section 43.
15. Germain D P, Waldek S, Banikazemi M, Bushinsky D A, Charrow J, et al. (2007) Sustained, long-term renal stabilization after 54 months of agalsidase beta therapy in patients with Fabry disease. J Am Soc Nephrol 18: 1547-1557.
16. Bishop D F, Desnick R J (1981) Affinity purification of alpha-galactosidase A from human spleen, placenta, and plasma with elimination of pyrogen contamination. Properties of the purified splenic enzyme compared to other forms. J Biol Chem 256: 1307-1316.
17. Kusiak J W, Quirk J M, Brady R O (1978) Purification and properties of the two major isozymes of alpha-galactosidase from human placenta. J Biol Chem 253: 184-190.
18. Lusis A J, Paigen K (1976) Properties of mouse alpha-galactosidase. Biochim Biophys Acta 437: 487-497.
19. Dooley S R (2014) Investigation of the Effect of Dimerization on Human α-Galactosidase Activity [Master of Science (M.S.), University of Massachusetts—Amherst].
20. Kizhner T, Azulay Y, Hainrichson M, Tekoah Y, Arvatz G, et al. (2015) Characterization of a chemically modified plant cell culture expressed human alpha-Galactosidase-A enzyme for treatment of Fabry disease. Mol Genet Metab 114: 259-267.
21. Grubb J H, Vogler C, Levy B, Galvin N, Tan Y, et al. (2008) Chemically modified beta-glucuronidase crosses blood-brain barrier and clears neuronal storage in murine mucopolysaccharidosis VII. Proc Natl Acad Sci USA 105: 2616-2621.
22. Ioannou-Y. A. (1990) Expression and Characterization of Recombinant Human Alpha-Galactosidase A: The City University of New York. 132 p.
23. Ausubel F R (1987) Current Protocols in Molecular Biology. John Wiley & Sons. pp. 8.3.
24. Chen Y, Jin M, Egborge T, Coppola G, Andre J, Calhoun D H, Expression and characterization of glycosylated and catalytically active recombinant human alpha-galactosidase a produced in pichia pastoris. Protein Expr Purif (2000) 20: 472-84. doi: 10.1006/prep.2000.1325. pmid:11087687.
25. Yasuda K, Chang H H, Wu H L, Ishii S, Fan J Q, Efficient and rapid purification of recombinant human alpha-galactosidase a by affinity column chromatography. Protein Expr Purif (2004) 37: 499-506. doi: 10.1016/j.pep.2004.07.005. pmid:15358377.
26. Gomori G, Preparation of buffers for use in enzyme studies. Methods Enzymol (1955) 1:138-146. doi: 10.1016/0076-6879(55)01020-3.
27. Zhang W, Mehmet I, Meagher M M, Fermentation strategies for recombinant protein expression in the methylotrophic yeast pichia pastoris. Biotechnol Bioprocess Eng (2000) 5:275-287. doi: 10.1007/BF02942184.

### INCORPORATION OF SEQUENCE LISTING

Incorporated herein by reference in its entirety is the Sequence Listing for this application. The Sequence Listing is disclosed on a computer-readable ASCII text file titled, “1038155_ST25.txt”, created on Aug. 22, 2016. The Sequence Listing .txt file is 7.57 KB in size.

1. A modified alpha-galactosidase A polypeptide comprising: SEQ ID NO. 1 having at least one mutation selected from the group comprising E203C/N, Y207R/W, Y134W, and R227W; or a functional homologue thereof; or functional fragments thereof.

2. The modified alpha-galactosidase A polypeptide according to claim 1, wherein the mutations comprise E203C/N and Y207R/W; or a functional homologue thereof; or functional fragments thereof.

3. The modified alpha-galactosidase A polypeptide according to claim 1, wherein the mutations comprise E203C and Y207W; or a functional homologue thereof; or functional fragments thereof.

4. The modified alpha-galactosidase A polypeptide according to claim 1, wherein the mutations comprise Y207W; or a functional homologue thereof; or functional fragments thereof.

5. A modified alpha-galactosidase A polypeptide of SEQ ID NO. 1 having at least one mutation selected from the group comprising E48C, L275C, R301C, H302C, R49C, S304C, G361C, P362C, N408C, P409C, D234C, S235C, N272C, F273C, G274C, S276C, W277C, E358C, I359C, and G360C; or a functional homologue thereof; or functional fragments thereof.

6. A modified alpha-galactosidase A polypeptide according to claim 5, wherein the mutations comprise N272C, F273C, G274C, S276C, and W277C; or a functional homologue thereof; or functional fragments thereof.

7. A modified alpha-galactosidase A polypeptide having a first mutation comprising at least one of E203C/N, Y207R/W, Y134W, and R227W; and a second mutation comprising at least one of E48C, L275C, R301C, H302C, R49C, S304C, G361C, P362C, N408C, P409C, D234C, S235C, N272C, F273C, G274C, S276C, W277C, E358C, I359C, and G360C; or a functional homologue thereof; or functional fragments thereof.

8. A stabilized alpha-galactosidase A dimer comprising:
a dimeric protein comprising monomers comprising an alpha-galactosidase A polypeptide having at least one mutation selected from the group comprising : E203C/N, Y207R/W, Y134W, R227W, E48C, L275C, R301C, H302C, R49C, S304C, G361C, P362C, N408C, P409C, D234C, S235C, N272C, F273C, G274C, S276C, W277C, E358C, I359C, and G360C; or a functional homologue thereof; or functional fragments thereof;
wherein each monomer of the dimer is covalently linked by at least one disulfide bond.

9. A stabilized alpha-galactosidase A dimer according to claim 7, wherein the mutation comprises E203C/N, Y207R/W, Y134W, and R227W; or a functional homologue thereof; or functional fragments thereof.

10. A stabilized alpha-galactosidase A dimer according to claim 7, wherein the mutation comprises E48C, L275C, R301C, H302C, R49C, S304C, G361C, P362C, N408C, P409C, D234C, S235C, N272C, F273C, G274C, S276C, W277C, E358C, I359C, and G360C; or a functional homologue thereof; or functional fragments thereof.

11. A stabilized alpha-galactosidase A dimer according to claim 7, wherein the polypeptide comprises two mutations; wherein the first mutation comprises at least one of E203C/N, Y207R/W; and the second mutation comprises at least one of N272C, F273C, G274C, S276C, and W277C; or a functional homologue thereof; or functional fragments thereof.

12. A stabilized alpha-galactosidase A dimer according to claim 7, wherein the polypeptide comprises two mutations; wherein the first mutation comprises Y207W; and the second mutation comprises at least one of N272C, F273C, G274C, S276C, and W277C; or a functional homologue thereof; or functional fragments thereof.

13. The stabilized alpha-galactosidase A dimer according to claim 7, wherein at least one dimer is covalently linked to at least one other dimer; or a functional homologue thereof; or functional fragments thereof.


SEQUENCE LISTING (3 sequences)

SEQ ID NO: 1 - protein, 429 aa, Homo sapiens
MQLRNPELHLGCALALRFLALVSWDIPGARALDNGLARTPTMGWLHWERFMCNLDCQEEPDSCISEKLFMEMAELMVSEGWKDAGYEYLCIDDCWMAPQRDSEGRLQADPQRFPHGIRQLANYVHSKGLKLGIYADVGNKTCAGFPGSFGYYDIDAQTFADWGVDLLKFDGCYCDSLENLADGYKHMSLALNRTGRSIVYSCEWPLYMWPFQKPNYTEIRQYCNHWRNFADIDDSWKSIKSILDWTSFNQERIVDVAGPGGWNDPDMLVIGNFGLSWNQQVTQMALWAIMAAPLFMSNDLRHISPQAKALLQDKDVIAINQDPLGKQGYQLRQGDNFEVWERPLSGLAWAVAMINRQEIGGPRSYTIAVASLGKGVACNPACFITQLLPVKRKLGFYEWTSRLRSHINPTGTVLLQLENTMQMSLKDLL

SEQ ID NO: 2 - DNA, 1290 bp, Homo sapiens
atgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaa

SEQ ID NO: 3 - DNA, 1418 bp, Homo sapiens
aaacaataacgtcattatttaataagtcatcggtgattggtccgcccctgaggttaatcttaaaagcccaggttacccgcggaaatttatgctgtccggtcaccgtgacaatgcagctgaggaacccagaactacatctgggctgcgcgcttgcgcttcgcttcctggccctcgtttcctgggacatccctggggctagagcactggacaatggattggcaaggacgcctaccatgggctggctgcactgggagcgcttcatgtgcaaccttgactgccaggaagagccagattcctgcatcagtgagaagctcttcatggagatggcagagctcatggtctcagaaggctggaaggatgcaggttatgagtacctctgcattgatgactgttggatggctccccaaagagattcagaaggcagacttcaggcagaccctcagcgctttcctcatgggattcgccagctagctaattatgttcacagcaaaggactgaagctagggatttatgcagatgttggaaataaaacctgcgcaggcttccctgggagttttggatactacgacattgatgcccagacctttgctgactggggagtagatctgctaaaatttgatggttgttactgtgacagtttggaaaatttggcagatggttataagcacatgtccttggccctgaataggactggcagaagcattgtgtactcctgtgagtggcctctttatatgtggccctttcaaaagcccaattatacagaaatccgacagtactgcaatcactggcgaaattttgctgacattgatgattcctggaaaagtataaagagtatcttggactggacatcttttaaccaggagagaattgttgatgttgctggaccagggggttggaatgacccagatatgttagtgattggcaactttggcctcagctggaatcagcaagtaactcagatggccctctgggctatcatggctgctcctttattcatgtctaatgacctccgacacatcagccctcaagccaaagctctccttcaggataaggacgtaattgccatcaatcaggaccccttgggcaagcaagggtaccagcttagacagggagacaactttgaagtgtgggaacgacctctctcaggcttagcctgggctgtagctatgataaaccggcaggagattggtggacctcgctcttataccatcgcagttgcttccctgggtaaaggagtggcctgtaatcctgcctgcttcatcacacagctcctccctgtgaaaaggaagctagggttctatgaatggacttcaaggttaagaagtcacataaatcccacaggcactgttttgcttcagctagaaaatacaatgcagatgtcattaaaagacttactttaaaatgtttattttattgcc